(19) United States
(12) Patent Application Publication
(10) Pub. No.: US 2002/0138136 A1
(43) Pub. Date: Sep. 26, 2002

(54) Medical device having radio-opacification and barrier layers

(75) Inventors: Verivada Chandru Chandresekaran, Mercer Island, WA (US); Graig Kveen, Maple Grove, MN (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SCIMED Life Systems, Inc.

(21) Appl. No.: 09/815,892

(22) Filed: Mar. 23, 2001

(51) Int. Cl. A61F 2/06

(52) U.S. Cl. 623/1.34

(57) ABSTRACT

A medical device such as a coronary stent is provided that can be visualized in-vivo while further aiding in the prevention of restenosis. The medical device comprises a core having a first layer disposed thereon. The first layer is made from a material that is radio-opaque so that the medical device may be visualized in-vivo. An outer layer is disposed onto and surrounds at least a portion of the first layer to provide a barrier layer between the radio-opaque inner layer and blood and/or tissue disposed within the patient's vessel. The outer surface of the outer layer may include a textured surface of micro-pores, grooves, cross-hatched lines to receive a therapeutic agent. Drugs and treatments which utilize anti-thombogenic agents, and anti-proliferation agents may be readily deployed from the textured outer surface of the outer layer of the medical device.

### FIELD OF THE INVENTION

The present invention relates generally to devices for preventing vascular diseases, and more specifically to in-vivo stents used in medical procedures.

### BACKGROUND OF THE INVENTION

As an alternative to vascular surgery, percutaneous transluminal angioplasty (PTA) and percutaneous transluminal coronary angioplasty (PTCA) procedures are being widely used for treating stenotic atherosclerotic regions of a patient's vasculature to restore adequate blood flow. Catheters having an expansible distal end, typically in the form of an inflatable balloon, are positioned in a vessel, such as a coronary artery, at a stenotic site. The expansible end is then expanded to dilate the vessel in order to restore adequate blood flow to regions beyond the stenosis. While PTA and PTCA have gained wide acceptance, these angioplasty procedures suffer from two major problems: abrupt closure and restenosis.

Abrupt closure refers to rapid re-occlusion of the vessel immediately after or within hours of the initial treatment, and often can result in myocardial infarction if blood flow is not restored in a timely manner. Abrupt closure often results from either an intimal dissection or from rapid thrombus formation which occurs in response to injury of the vascular wall from the initial angioplasty procedure. Restenosis refers to a re-narrowing of the artery over the weeks or months following an initially apparently successful angioplasty procedure. Restenosis occurs in a significant amount of all angioplasty patients and results, at least in part, from smooth muscle cell proliferation and migration.

Many different strategies have been proposed to diminish the likelihood of abrupt closure and reduce the rate of restenosis. One such method involves the implantation of a vascular stent following angioplasty. Stents are thin-walled tubular scaffolds, which are expanded in the arterial lumen following the angioplasty procedure. Most commonly, the stents are formed from a malleable material, such as stainless steel, and are expanded in-situ using a balloon. Alternatively, the stents may be formed from a shape memory alloy or other elastic material, in which case they are allowed to self-expand at the angioplasty treatment site. In either case, the stent acts as a mechanical support for the artery wall, thereby inhibiting abrupt closure and reducing the restenosis rate as compared to PTCA.

Recent developments in medical devices have stressed the importance of visually perceiving the stent in-vivo as it is being placed within the vasculature of the patient. Additionally, it is advantageous and sometimes necessary to visually locate and inspect a previously deployed stent or to treat restenosis occurring at the location of the stent. Fluoroscopy is one technique that allows visualization of a stent in-vivo. To visualize the stent in-vivo using fluoroscopy, the stent must be made from a material that is highly radio-opaque or must use a delivery catheter that provides radio-opaque markers. However, the preferred structural material, stainless steel, used in stents is not highly radio-opaque. Thus, several solutions have been proposed such as coating a conventional stainless steel stent with a radio-opaque material such as gold.

While coated and non-coated stents have been successful in inhibiting abrupt closure and reasonably successful in inhibiting restenosis, a significant portion of the treated patient population still experiences restenosis over time. It is possible for the alloying metals of the stent material (e.g. stainless steel) or the gold alloy coating to be leached by the body fluids resulting in the activation of platelets and cells, the possible precursor to thrombus formation, on a localized level. Additionally, most stent structures comprise an open lattice, typically in a diamond or spiral pattern, and cell proliferation (also referred to as intimal hyperplasia) can intrude through the interstices between the support elements of the lattice and the treatment site once again becomes occluded.

Therefore, there is a need for an improved medical device that can be visualized in-vivo while further aiding in the prevention of restenosis.

### SUMMARY OF THE INVENTION

The present invention addresses the need for an improved medical device that can be visualized in-vivo while further aiding in the prevention of restenosis by providing a medical device having radio-opacification and at least one barrier layer.

In accordance with a first aspect of the present invention, a laminate structure is provided for making a medical device. The laminate structure comprises a core having an outer surface and a first layer secured onto a portion of the outer surface of the core. The first layer has an outer surface and is radio-opaque. A second biocompatible layer is secured onto at least a portion of the outer surface of the first layer to reduce contact between the first layer and blood and/or tissue in a vessel.

In accordance with another aspect of the present invention, the outer surface of the second layer has micro-pores or other structures to receive therapeutic drugs and deliver them to the vessel in the area of the medical device.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates a side view of a conventional medical device;

FIG. 2 illustrates a side view of a medical device in accordance with an embodiment of the present invention;

FIG. 3 illustrates a cross-sectional view taken along lines A-A of the medical device shown in FIG. 2;

FIG. 4 illustrates a magnified portion of the cross-sectional view taken along lines A-A of the medical device shown in FIG. 2;

FIG. 5 illustrates a cross-sectional view of a portion of a medical device according to a second embodiment of the present invention;

FIG. 6 illustrates a cross-sectional view of a portion of a medical device according to a third embodiment of the present invention;

FIG. 7 illustrates a cross-sectional view of a medical device in-situ in a patient's vessel according to a fourth embodiment of the present invention;

FIG. 8 illustrates a cross-sectional view of a medical device in-situ in a patient's vessel according to a fifth embodiment of the present invention;

FIG. 9 illustrates a cross-sectional view of a medical device in-situ in a patient's vessel according to a sixth embodiment of the present invention; and

FIG. 10 illustrates a cross-sectional view of a portion of a medical device having a circular cross-section.

### DETAILED DESCRIPTION OF THE PREFERRED EMBODIMENT

While, as will be better understood from the following description, the present invention was developed for coronary stents and, thus, is expected to find its primary use with such coronary stents, it is to be understood that the invention can be used with other medical devices such as vena cava filters, aneurysm coils or other implantable devices that require the ability to be visualized in-vivo and to have a biocompatible barrier layer. Thus, it is to be understood that the disclosed embodiment is only by way of example and should not be construed as limiting.

Prior to describing an illustrative embodiment of the invention, a brief discussion of the structure of one type of medial device is set forth. In this regard, attention is directed to FIG. 1, which illustrates a conventional medical device known in the art as a coronary stent 10. The coronary stent 10 is deployed in-vivo at a stenosed vessel following a PTCA procedure. The stent 10 is deployed from a delivery catheter just proximal to the diseased section of the vessel and is expanded into abutment against the interior lining of the vessel wall. Once in-situ, the stent 10 acts as a mechanical support for the vessel wall, inhibiting abrupt closure.

Referring again to FIG. 1, the skeletal frame of the stent 10 preferably includes wire or bar-like members 12, each forming a distinct, repetitive zigzag pattern. This repetitive zigzag pattern consists of multiple V-shaped curves 14. The areas 16 within the V-shaped curves 14 are open. With no recognizable beginning or end to this zigzag pattern, the bar-like member 12 forms expandable zigzag segment 18. A plurality of zigzag segments 18 are arranged along the longitudinal axis of the stent 10 so that the V-shaped curves 14 of abutting zigzag segments 18 may be joined through an interconnecting element 20. Through the interconnecting elements 20, a continuous wire-like framework is created between the multiple zigzag elements 18 forming the stent 10.

The coronary stent illustrated in FIG. 1 is only exemplary of many of the various medical devices which may incorporate the benefits of the present invention. The present invention could also be used with devices such as vena cava filters or aneurysm coils and other small implanted devices that need to be fluoroscopically visible. For clarity, the remaining detailed description refers only to a stent. However, it will be appreciated that any medical device can incorporate the aspects of the present invention. The method of making and using the stents described above and used in conjunction with PTCA procedures are well known in the art and are not described in detail here.

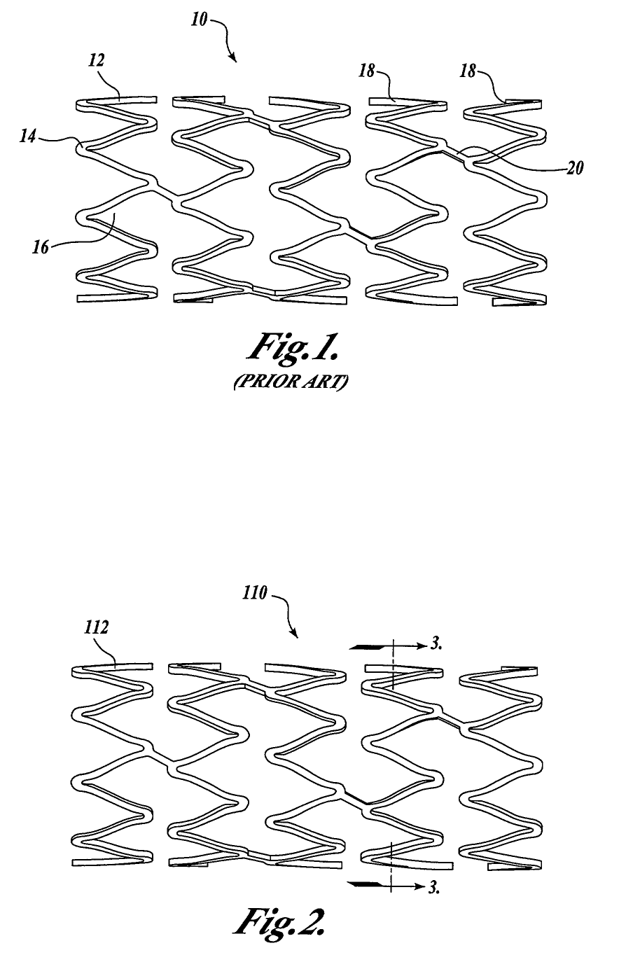

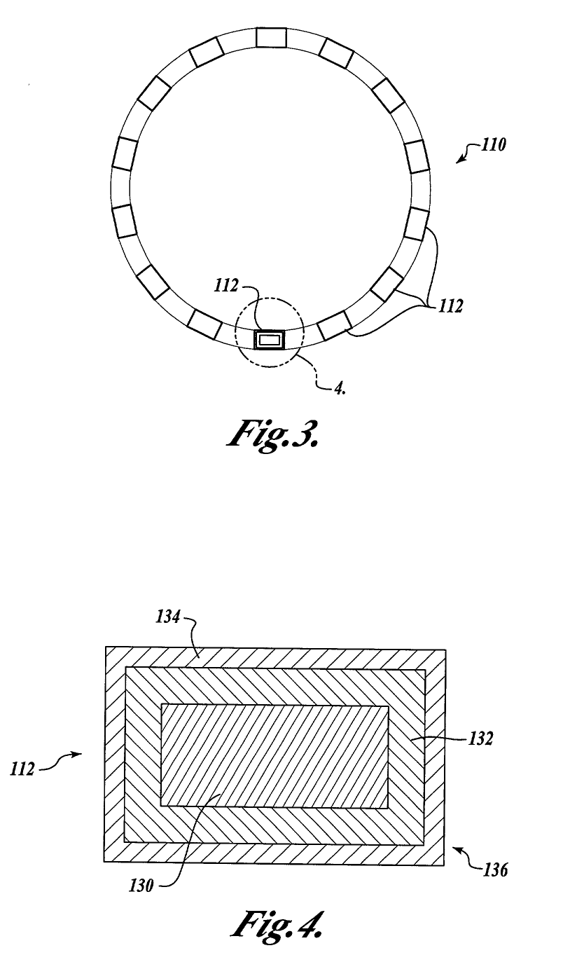

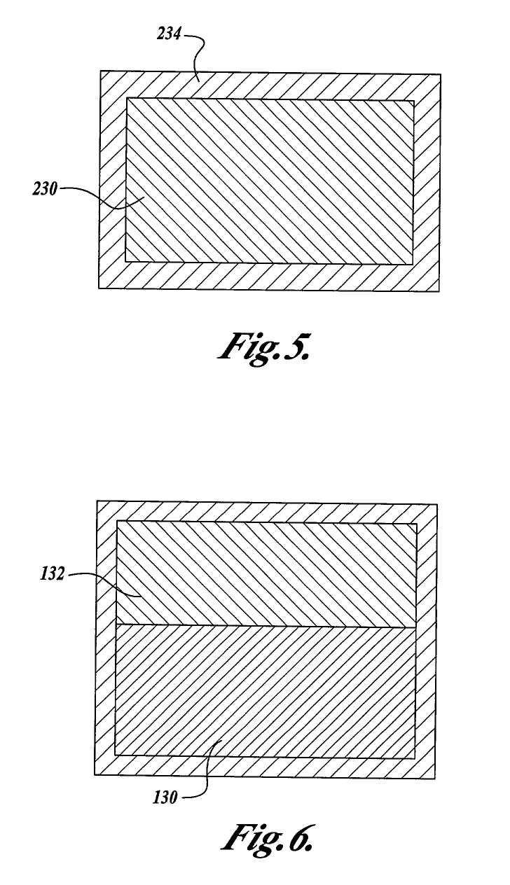

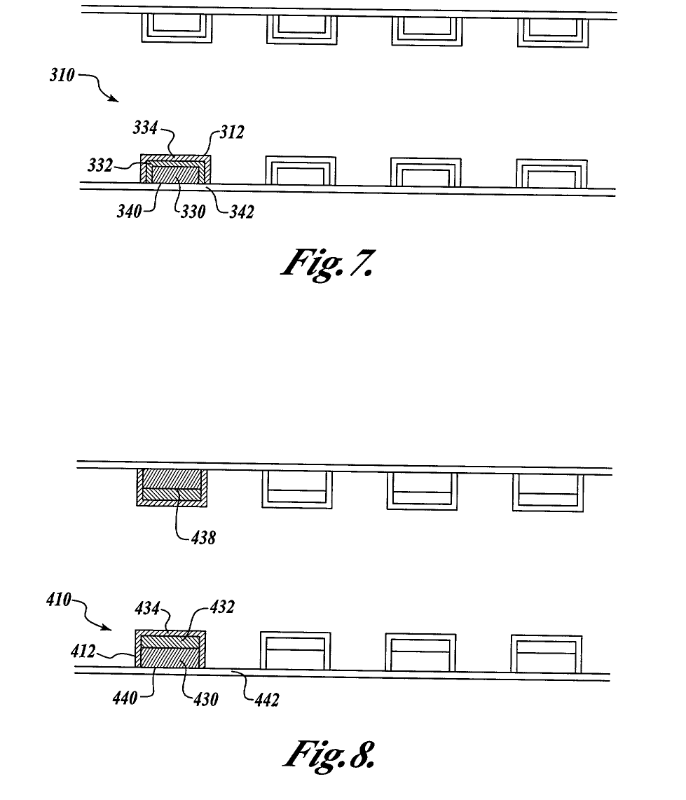

The present invention is directed to an improved coronary stent that provides in-vivo visualization and a bio-compatible barrier layer that may reduce the possibility of restenosis. These characteristics are attributable to constructing the coronary stent with a laminate or composite structure. FIGS. 2-3 illustrates an exemplary embodiment of the improved stent 110 constructed in accordance with the aspects of the present invention. The stent 110 is comprised of many bar-like members 112. As best shown in FIG. 4, the members 112 when viewed in cross-section include a core or body 130, and a first or inner layer 132 disposed directly adjacent to and preferably surrounding the core 130. However, it will be appreciated that other configurations of the inner layer may be utilized. For example, as best shown in FIG. 6, the inner layer 132 may be disposed on one side of the core 130.

The core 130 is constructed from a material that provides the stent with the necessary strength and flexibility to support the diseased vessel. The core 130 is preferably made from 316 stainless steel; however, other materials may be used such as titanium, nickel titanium, or tantalum or their alloys. In an alternative embodiment, the core 130 can include a centrally located lumen extending longitudinally therethrough, instead of being of a solid construction, as shown in FIG. 4. The inner layer 132 disposed over the core is constructed from a radio-opaque material that permits fluoroscopic imaging and is magnetic resonance imaging (MRI) distortion free such as gold or a gold alloy of nickel, chromium, copper, or iron. It will be understood that the thickness of the inner layer is such (preferably 3-12 microns) that it can be viewable during fluoroscopy.

Disposed over the inner layer 132 is an outer layer 134 that forms the outermost surface of the stent. The outer layer 134 overlays the inner layer 132 to form a barrier between the inner layer and the blood and/or tissue of the patient's vessel. Additionally, the outer layer 134 provides a dielectric barrier that inhibits charge transfer to and from the inner layer 132. Through the multiple layers of the core 130, inner layer 132, and outer layer 134, a laminate or composite structure 136 is constructed to form the members 112. The members 112 may be arranged in a variety of configurations to form the stent 110.

The outer layer 134 is made from a bio-compatible or “bio-friendly” material that is chemically inert with human blood and tissue and preferably has a thickness of approximately one micron. The outer layer is chemically inert from its inherent ability to form a stable oxide or nitride. The oxide or nitride forms a thin film on the outer surface of the outer layer to form a protective barrier. Some examples of suitable materials that may be used for the outer layer include, but are not limited to stainless steel, titanium (Ti), chromium (Cr), tantalum (Ta), aluminum (Al), and vanadium (V), all of which form stable oxides in the native form or are induced by thermal oxidation. Stainless steel may also be suitably passivated to form a robust oxide. Likewise, nitrides of the same materials can be used as the outer layer and are formed in a plasma reactor. Other suitable complexes such as carbides, oxy-nitrides, and suicides may be also used based on their relative compatibility with blood and tissue. Further, any bio-compatible polymer may be used. The outer layer 134 may also include platinum, irridium and their alloys. Regardless of the material used, it is preferable to use one that is MRI distortion free.

FIG. 5 illustrates another exemplary embodiment of the stent according to the present invention. The stent comprises a core 230 having an outer layer 234 disposed thereon. The core 230 is preferably comprised of an alloy of gold and titanium or tantalum or combinations thereof. Other materials having the necessary requirements of strength and radio-opacity may also be utilized to form the core 230. For example, the core can be composed of an alloy consisting of 70% gold and 30% titanium. The outer layer 234, made from any suitable bio-compatible material described above, is then plated onto the core 230 to provide a barrier between the alloy and the patient's blood and/or tissue. Alternatively, the core and outer layer may be bonded together by co-extrusion or rolling and the stent is fabricated from this laminate composite.

FIG. 7 illustrates a cross-sectional view of a stent in-situ in a patient's vessel according to yet another exemplary embodiment of the present invention. The stent 310 is comprised of multiple bar-like members 312. The members 312 include a rectangular shaped core or body 330, a radio-opaque inner layer 332 disposed on a portion of the core 330, and an outer layer 334 that overlays the radio-opaque inner layer 332 to form a laminate or composite structure. The bottom surface 340 of the core 330, which is left uncovered by the inner layer 332, engages the vessel wall 342 when the stent is in-situ. The outside layer 334 provides a barrier between the radio-opaque inner layer 332 and the blood within the patient's vessel. Any suitable material, as discussed above with reference to FIG. 4, may be used for each layer of the laminate structure.

FIG. 8 illustrates a cross-sectional view of a stent in-situ in a patient's vessel according to yet another exemplary embodiment of the present invention. The stent 410 is comprised of multiple bar-like members 412. The members 412 include a rectangular shaped core or body 430, a radio-opaque inner layer 432 disposed on the top surface 438 of the core 430, and an outer layer 434 disposed over the inner layer 432 and a portion of the core 430 to form a laminate or composite structure. The bottom surface 440 of the core 430, which is left uncovered by the inner layer 432, engages the vessel wall 442 when the stent is in-situ. The outside layer 434 provides a barrier between the radio-opaque inner layer 432 and the blood within the patient's vessel. Additionally, the core 430 provides a barrier between the radio-opaque inner layer 432 and the vessel wall. Any suitable material, as discussed above with reference to FIG. 4, may be used for each layer of the laminate structure.

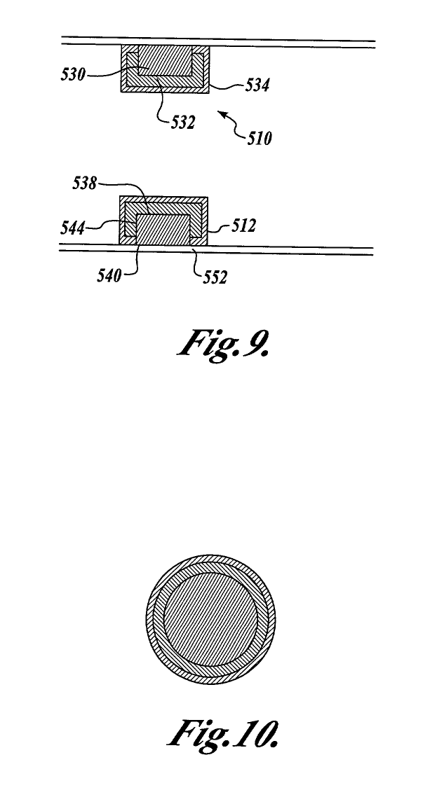

FIG. 9 illustrates a cross-sectional view of a stent in-situ in a patient's vessel according to still yet another exemplary embodiment of the present invention. The stent 510 is comprised of multiple bar-like members 512. The members 512 include a rectangular shaped core or body 530, a radio-opaque inner layer 532, and an outer layer 534 to form a laminate or composite structure. The inner layer 532 is disposed over the top surface 538 of the core and a portion 544 of the side surfaces of the core 530. The outer layer 534 overlays the inner layer 532 and the remaining portion of the side surfaces of the core 530. The bottom surface 540 of the core 530, which is left uncovered by the inner layer 532, engages the vessel wall 552 when the stent is in-situ. The outside layer 534, in conjunction with the core 530, provides a barrier between the radio-opaque inner layer 532 and the blood and/or tissue within the patient's vessel. Any suitable material, as discussed above with reference to FIG. 4, may be used for each layer of the laminate structure.

It will be appreciated by those skilled in the art that the laminate or composite structure that forms the stent illustrated in FIGS. 3-9 can be fabricated by various methods know in the art. For example, the inner layer may be disposed onto the core using conventional plating methods such as electro and/or electroless plating. Likewise, the outer layer may be disposed onto the inner layer by conventional plating methods. Other methods of disposing or bonding the layers onto the core can be used such as chemical vapor deposition and physical deposition in conjunction with selective masking, wet-chemical processing, and sol gel processing. Alternatively, separate sheets or tubes of material corresponding to the core and the inner and outer layers, respectively, can be fabricated into the laminate or composite structure by rolling (roll bonding) or co-extruding, or a combination of co-extruding, rolling, and plating. Those skilled in the art will appreciate that additional manufacturing processes such as annealing or electro-polishing may be administered during the fabrication of the composite structure to control the microstructure, internal stresses, composition and surface finish. Additionally, it will be appreciated by those skilled in the art that the outer layer can be fabricated to have a crystallographic structure that minimizes surface energy to reduce chemical and biochemical reactions at the surface of the outer layer.

Often it is beneficial to treat the localized area of the diseased vessel that is stented. The outer layer may include a textured surface of micro-pores, grooves, cross-hatched lines or the like to receive a therapeutic agent. Drugs and treatments which utilize anti-thrombogenic agents, and anti-proliferation agents may be readily deployed from the textured outer surface of the outer layer of the stent. Specific examples of preferred therapeutic agents include Taxol and Heparin. However, it is to be understood that other agents may be deployed. Additionally, the cellular response can be regulated with a suitable textured surface even in the absence of drugs. To this end, the textured surface of the outer layer of the stent may induce favorable biological reactions within the patient's vessel.

In conjunction with the various embodiments of the present invention, it will be appreciated by those skilled in the art that the gold alloy composition used for the inner layer can be varied throughout the thickness of the deposit to achieve specific mechanical properties such as flexibility, strength, and weight. For example, the density of the gold layer may fluctuate as it extends circumferentially around the core and as it extends outwardly from the core.

While the preferred embodiment of the invention has been illustrated and described, it will be appreciated that various changes can be made therein without departing from the spirit and scope of the invention. For example, it is contemplated to be within the scope of the invention to have a stent provided that already has been coated with a gold layer. The gold coated stent may then be plated with any suitable bio-compatible material discussed above to form a barrier between the gold plating and the blood and tissue within the patient's vessel. Additionally, the stent members are shown in FIGS. 2-9 as having a rectangular cross-section. However, it will be appreciated by those skilled in the art that other cross-sectional shapes may be utilized to provide the desired mechanical characteristics to the stent, such as a circular core, which is shown in FIG. 10, or elliptical. The stent members formed by these other cross-sectional shapes may also include a centrally located lumen extending longitudinally therethough, as described above with the exemplary embodiment shown in FIG. 4.

The embodiments of the invention in which an exclusive property or privilege is claimed are defined as follows:

1. A laminate structure for making a medical device comprising:
an core having an outer surface;
a first radio-opaque layer disposed on at least a portion of the outer surface of the core, the first radio-opaque layer having an outer surface; and
a second layer disposed on at least a portion of the outer surface of the first radio-opaque layer; wherein the second layer isolates the first radio-opaque layer from blood within a patient's vessel.

2. The laminate structure of claim 1, wherein the second layer covers a portion of the first radio-opaque layer and a portion of the core.

3. The laminate structure of claim 1, wherein the first radio-opaque layer surrounds the core.

4 The laminate structure of claim 3, wherein the second layer surrounds the first radio-opaque layer.

5. The laminate structure of claim 1, wherein the outer surface of the second layer is capable of receiving a drug compound.

6. The laminate structure of claim 1, wherein the second layer is made from an oxide of a metal selected from the group consisting of Ti, Cr, Ta, and Al.

7. The laminate structure of claim 1, wherein the second layer is made from a nitride of a metal selected from the group consisting essentially of Ti, Cr, Ta, and Al.

8. The laminate structure of claim 1, wherein the second layer is made from a carbide of a metal selected from the group consisting essentially of Ti, Cr, Ta, and V.

9. In a medical device implantable within a patient's vessel, the medical device including a core having an outer surface, the outer surface having a layered structure thereon, the layered structure comprising:
a radio-opaque inner layer disposed onto the outer surface of the core, and
an outer bio-compatible layer surrounding the radio-opaque inner layer;
wherein the outer layer isolates the radio-opaque inner layer from blood or tissue within the patient's vessel.

10. A medical device comprising:
a core having an outer surface;
a radio-opaque inner layer disposed onto at least a portion of the outer surface of the core, and
a bio-compatible outer layer, the outer layer covering at least a portion of the radio-opaque inner layer to reduce contact between the radio-opaque material and blood within a patient's vessel.

11. The medical device of claim 10, wherein the radio-opaque inner layer surrounds the core.

12. The medical device of claim 11, wherein the outer layer surrounds the radio-opaque inner layer to inhibit the radio-opaque layer from coming into contact with blood and tissue from with a patient's vessel.

13. The medical device of claim 10, wherein the medical device is a coronary stent.

14. The medical device of claim 10, wherein the outer layer is made from an oxide of a metal selected from the group consisting of Ti, Cr, Ta, and Al.

15. The medical device of claim 10, wherein the outer layer is made from a nitride of a metal selected from the group consisting essentially of Ti, Cr, Ta, and Al.

16. The medical device of claim 10, wherein the outer layer is made from a carbide of a metal selected from the group consisting essentially of Ti, Cr, Ta, and V.

17. A method of treating an occluded vessel with a stent, comprising the acts of:
routing a delivery catheter having the stent mounted or restrained thereon to a position proximal to the diseased section of the vessel wherein the stent is of the type that includes: a core having an outer surface, a radio-opaque inner layer disposed onto at least a portion of the outer surface of the core, and a bio-compatible outer layer, the outer layer covering at least a portion of the radio-opaque inner layer to reduce contact between the radio-opaque material and blood or tissue within the diseased vessel;
deploying the stent from the delivery catheter;
expanding the stent into abutment against the interior lining of the diseased vessel so as to provide a support mechanism to prevent closure of the vessel.

18. A device used in-vivo comprising:
a core;
means for increasing the visibility of the core to in-vivo viewing methods; and
means for establishing a barrier on the outer surface of the device so that the visibility increasing means is isolated from a patient's blood.

19. The device of claim 18, wherein the visibility increasing means comprises a radio-opaque layer disposed on at least a portion of the outer surface of the core.

20. The device of claim 19, wherein the means for establishing a barrier on the outer surface of the device comprises an outer layer disposed on at least a portion of the outer surface of the radio-opaque layer to form a barrier layer between the radio-opaque layer and the patient's blood.

21. The device of claim 20, wherein the outer layer is made from an oxide of a metal selected from the group consisting of Ti, Cr, Ta, and Al.

22. The device of claim 20, wherein the outer layer is made from a nitride of a metal selected from the group consisting essentially of Ti, Cr, Ta, and Al.

23. The device of claim 20, wherein the outer layer is made from a carbide of a metal selected from the group consisting essentially of Ti, Cr, Ta, and V.
